(19) United States
(12) Patent Application Publication
(10) Pub. No.: US 2025/0345476 A1
(43) Pub. Date: Nov. 13, 2025

(54) DEVICE FOR DISCHARGING A DISINFECTING SOLUTION

(71) Applicant: ATC SOLUTIONS GmbH, Worb (CH)

(72) Inventor: Ibrahim BEN, Worb (CH)

(73) Assignee: ATC SOLUTIONS GmbH, Worb (CH)

(21) Appl. No.: 18/658,731

(22) Filed: May 8, 2024

(51) Int. Cl. A61L 2/26; A61L 2/18

(52) U.S. Cl. CPC A61L 2/26; A61L 2/18; A61L 2202/123; A61L 2202/15

(57) ABSTRACT

A device for discharging a disinfecting solution to disinfect surfaces is configured to be arrangeable on a carrier surface and includes an exchangeable receptacle (39′) for receipt of the disinfecting solution, a housing enclosing the receptacle, a spray head (30′) configured to discharge disinfecting solution from the receptacle, and a pumping equipment for transporting disinfecting solution from the receptacle to the spray head and for discharging the disinfecting solution from the spray head. In another aspect, there is provided a spray including an inner nozzle part (50) and an outer nozzle part (51). The inner nozzle part includes a first channel for conducting disinfecting solution to a liquid outlet and a second channel for conducting pressurized air to an air outlet. The outer nozzle part includes a spray outlet and encloses the inner nozzle part to form a space between the air outlet und the spray outlet. The liquid outlet is arranged in the space to mix liquid, in particular disinfecting solution, ejecting out of the liquid outlet with pressurized air.

### BACKGROUND OF THE INVENTION

### Field of the Invention

The present invention relates to a device for discharging in particular a disinfecting solution to disinfect surfaces and to a spray head for such a device.

### Description of the Related Art

In different locations, such as hospitals and dentist practices, it is necessary to disinfect surfaces on a regular basis. Disinfection is laborious and time-consuming when done by hand. Automated disinfection is therefore preferred.

Here and in the following description and claims the term disinfecting solution is used for an agent used to inhibit or kill microorganisms to a certain degree.

The patent application US2021386892 A1 describes a room sanitization system that allows surfaces in a room to be treated with a disinfecting solution. The system has a plurality of sprinkler heads arranged in a room and connected via a tubing to a distant hub. This system is difficult to install and the flexibility in use is reduced. If a room is to be retrofitted additional tubing must be provided.

### SUMMARY OF THE INVENTION

It is an aim of the present invention to provide for a device for discharging in particular a disinfecting solution, which can be used flexibly to disinfect selected surfaces in a room.

This aim is achieved by a device as defined in the first independent claim.

It is another aim of the present invention to provide for a spray head which allows an efficient discharge of a liquid, in particular a disinfecting solution.

This aim is achieved by a spray head as defined in the second independent claim.

The further claims specify preferred embodiments of the device and of the spray head and a further device for discharging a liquid.

The device according to the invention is configured to be arrangeable on a carrier surface. The surface may be oriented for instance horizontally, e.g. as being part of a table or ceiling, or vertically, e.g. as being part of a wall.

The device may comprise a base configured to be attached, e.g. by means of screws, on a ceiling or wall.

The device and the spray head may be configured such that a disinfecting solution or another liquid may be discharged.

The device according to the invention comprises an exchangeable receptacle for receipt of a liquid, in particular a disinfecting solution, a spray head configured to discharge liquid from the receptacle, and a pumping equipment for transporting liquid from the receptacle to the spray head and for discharging it. The device may be configured as a stand-alone device and allows a flexible usage.

In one embodiment, the device includes a single spray head.

Preferably, the device comprises a base part arrangeable on the carrier surface and a rotary part, which includes the spray head and which is configured to be rotatable with respect to the base part.

Preferably, the rotary part is rotatable around a rotation axis over an angle range of at least 90 degrees and by order of increasing preference at least 180 degrees, at least 270 degrees or at least 360 degrees.

Preferably, the rotary part comprises the receptacle and/or an electric drive.

The base part of the device may comprise an internal toothing and the rotary part may comprise a drive gear meshing with the internal toothing.

The rotary part may comprise an electric drive to which the drive gear is coupled.

Preferably, the device comprises an electric transmission equipment configured to supply electrical power from the base part to the rotary part.

The electric transmission may e.g. comprise a rotary electric slip ring.

In one embodiment, the spray head is configured to be rotatable around a rotation axis, e.g. drivable by an electric drive, and to be swivelled around a swivel axis, e.g. manually. Thus, the spray head is movable around two axes. The swivel axis may be arranged transversely to the rotation axis. The spray head may be rotated around the rotation axis over an angle range of at least 90 degrees and by order of increasing preference at least 180 degrees, at least 270 degrees or at least 360 degrees. The spray head may be swivelled around the swivel axis over an angle range of at least 10 degrees and/or of less than 90 degrees.

Preferably, the receptacle of the device comprises a bottle including a threaded bottleneck and/or a bottleneck with a plug. The plug may comprise a through opening for extracting liquid, in particular disinfecting solution, and at least one hole for pressurizing the liquid, in particular disinfecting solution, in the bottle.

Preferably, the device comprises a connection port configured to be connected to the receptacle by means of a screw connection.

For instance, the connection port may be threaded for a removable connection with the threaded bottleneck of the receptacle.

Preferably, the connection port comprises a sealing equipment configured to provide an airtight connection with the receptacle.

Preferably, the connection port is arranged on top of the receptacle.

Preferably, the pumping equipment of the device comprises a pump to produce pressurized air. The pump may be configured such that it produces, at an ambient temperature of 20 degree Celsius, air with a pressure of at least 100 kPa, preferably at least 200 kPa, most preferably at least 250 kPa. The air pressure may be e.g. 280 kPa.

The pump may be configured such that, at an ambient temperature of 20 degree Celsius, it produces a volume flow of air of at least 10 litre per minute, preferably at least 20 litre per minute and most preferably at least 25 litre per minute. The volume flow of air may be e.g. 30 litre per minute.

Preferably, the device comprises a feed channel with a bifurcation to guide pressurized air to the receptacle and to the spray head.

Preferably, the spray head of the device comprises a first channel for conducting liquid, in particular disinfecting solution, to a liquid outlet and a second channel for conducting pressurized air to the liquid outlet to mix it with liquid, in particular disinfecting solution, ejecting out of the liquid outlet.

The liquid outlet may be part of a first nozzle, which is configured to receive the liquid, in particular disinfecting solution, from the receptacle and which is arranged in a second nozzle, in which pressurized air can be added.

The first nozzle may have a liquid outlet with a diameter of less than 1.0 mm, preferably less than 0.5 mm. The diameter may be e.g. in the range of 0.3 to 0.4 mm.

The second nozzle may have a spray outlet with a diameter of less than 4 mm, preferably less than 3 mm.

Preferably, the spray head comprises an inner nozzle part for receiving and ejecting liquid, in particular disinfecting solution, and an outer nozzle part for receiving pressurized air and mixing it to the liquid, in particular ejected disinfecting solution, wherein the inner nozzle part and the outer nozzle part are detachably connected to each other.

A seal may be arranged between the inner nozzle part and the outer nozzle. The inner nozzle part and the outer nozzle may be detachably connected e.g. by means of a screw connection.

Preferably, the device comprises a communication module configured to enable a wireless communication with a mobile phone for controlling the device.

Preferably, the housing of the device comprises a removable cover enabling an access to the receptacle for a replacement.

Preferably, the device comprises a power supply unit configured to supply the device with power and connectable to the mains via a cable.

### BRIEF DESCRIPTION OF THE DRAWINGS

The invention is explained in the following by means of exemplary embodiments with reference to Figures. In the drawings:

FIG. 1 shows a first embodiment of a device for discharging a disinfecting solution in a perspective view;

FIG. 2 shows the device of FIG. 1 in a first side view;

FIG. 3 shows the device of FIG. 1 in a second side view;

FIG. 4 shows the device of FIG. 1 in an explosive view;

FIG. 5 shows a second embodiment of a device for discharging a disinfecting solution in a top view;

FIG. 6 shows the device of FIG. 5 in a first side view;

FIG. 7 shows the device of FIG. 5 in a second side view;

FIG. 8 shows the device of FIG. 5 in a third side view;

FIG. 9 shows part of the upper portion of the device of FIG. 5 in a partly sectioned side view;

FIG. 10 shows a bottle containing a disinfecting solution to be received in a device of FIG. 5; and

FIG. 11 shows a detailed sideview of forward part of the spraying head of the device of FIG. 5.

### DETAILED DESCRIPTION

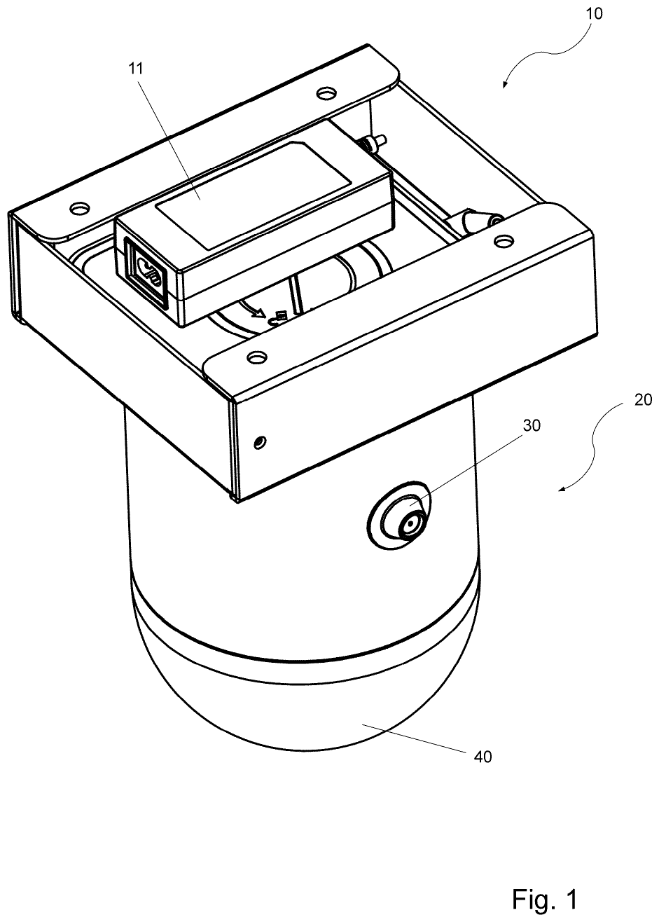

FIG. 1 shows a first embodiment of a device for discharging a disinfecting solution to disinfect surfaces. The device of this embodiment is configured to be arranged on a carrier surface in form of a ceiling and includes a base 10 to be mounted on the ceiling by using e.g. screws (not shown). The device includes a body part 20 which is configured to be rotatable relative to the base 10, which is stationary when mounted. The base 10 includes a power supply unit 11 which is connectable to the mains via a cable (not shown) and which serves for supplying the device with power, e.g. in form of a DC current.

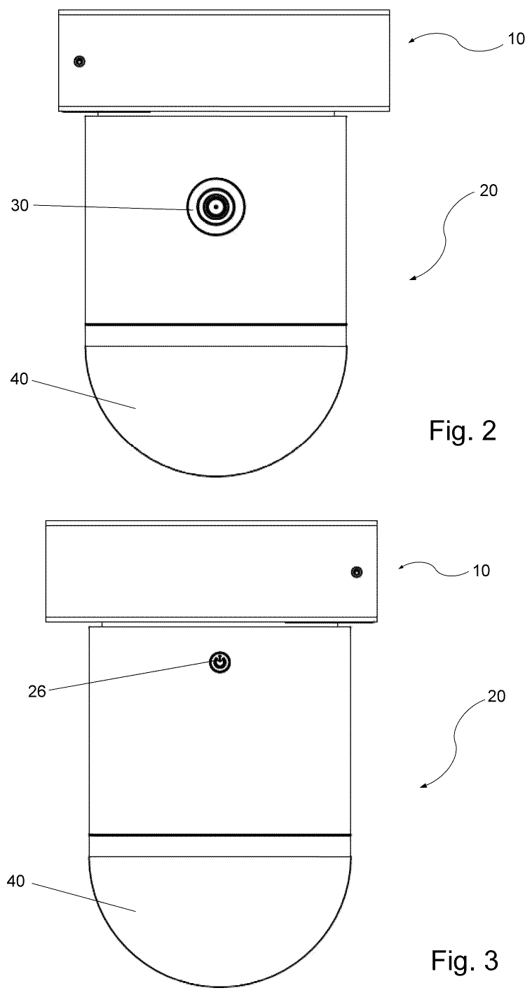

As is also apparent from FIG. 2, the body part 20 includes a spraying head 30 for discharging disinfecting solution to the outside.

As is apparent from FIG. 3, the device includes a control button 26 which is arranged in the present embodiment on the body part 20. The control button 26 serves for switching on and off the device. Further functions may be activated when operating the control button 26, e.g. a resetting of the device.

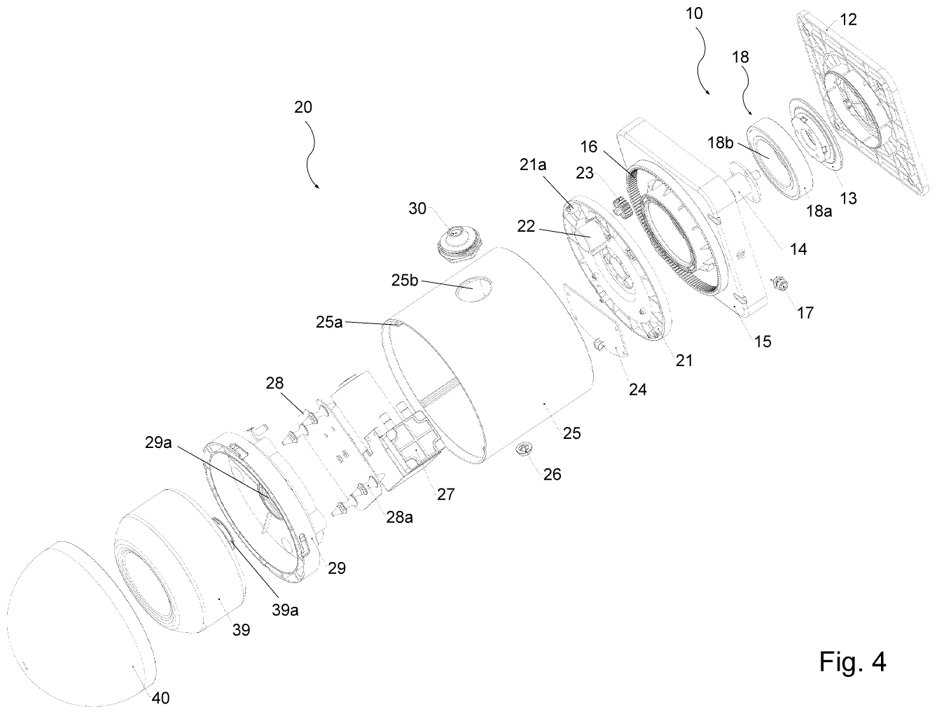

A cover 40 is arranged at the bottom of the body part 20 and is configured to be removable to reveal a receptable 39 containing a disinfecting solution, see FIG. 4.

FIG. 4 shows the structure of the base 10 and of the body part 20 in more detail. The base 10 includes a frame 15 which is provided with a circumferential internal toothing 16 and with an electrical connector 17, e.g. a DC terminal. The electrical connector 17 can be connected to the power supply unit 11. In order to supply electrical power from the stationary base 10 to the rotary body part 20, an electric transmission equipment including a stationary connection board 13 and a rotary electric slip ring 14 is provided.

The electrical connector 17 is connected to the connection board 13, from where electrical power can be transmitted to the electric slip ring 14.

The frame 15 is covered by a cover plate 12 to enclose a space for receiving a bearing 18. The latter is arranged coaxially to the internal toothing 16 and formed e.g. as a ball bearing which includes an outer ring 18a held in the base 10 and an inner ring 18b to which a carrier structure 21, 25, 29 of the body part 20 is attached. The carrier structure includes a rearward carrier 21 and a forward carrier 29, which are arranged at a respective side of a shell 25 to form a housing.

The shell 25 may be formed as a cylindrical element and may include e.g. rail elements 25a which run inside the shell 25 along its longitudinal direction. The carriers 21, 29 may include protrusions 21a configured to engage in the rail elements 25a.

A drive 22, e.g. a stepper motor, is attached to the rearward carrier 21. The rotary shaft of the drive 22 is provided with a drive gear 23 meshing with the internal toothing 16 of the base 10. A control unit 24 for controlling the device is attached to the rearward carrier 21. The control unit 24 includes e.g. a circuit board. The drive 22 and the control unit 24, which are part of the body part 20 and are therefore rotatable, can be energized by means of the power supply unit 11 via the electric slip ring 14.

The shell 25 may be formed of a suitable material, e.g. metal, in particular a metal including aluminium, e.g. an aluminium alloy, and includes openings 25b for receiving the control button 26 and the spraying head 30.

A pump 27 is arranged on a carrier plate 28 which is provided with dampening elements 28a, which are attached to the forward carrier 29. Here, the pump 27 is configured to produce high-pressure air, typically at least 100 kPa when measured at an ambient temperature of 20 degree Celsius, e.g. 280 kPa. Further, the pump is configured such that, at an ambient temperature of 20 degree Celsius, it produces a volume flow of air of at least 10 litre per minute. The volume flow of air may be e.g. 30 litre per minute.

The dampening elements 28a serve for decoupling the pump 27 from the rest of the device so that vibrations produced by the pump 28 during operation are transferred to a reduced extent and/or so that the pump 28 produces less noise during operation. The dampening elements 28a may be made from an elastic material, such as rubber. One or more dampening elements in form of a flat material, such as a silicone gasket, may be arranged between the pump 27 and the carrier plate 28 (not shown).

The forward carrier 29 includes a connection port 29a to which a receptacle 39 including a disinfecting solution can be attached. Here, the receptacle 39 is formed as a bottle with a threaded bottleneck 39a. The connection port 29a includes e.g. an inner thread, into which the bottleneck 39a can be screwed. The receptacle 39 is provided with a tube (not shown) which preferably reaches to the bottom of the receptacle 39. In use, disinfecting solution is sucked via the tube to the spraying head 30 by means of the pump 27. The receptacle 39 may be configured to be disposable, so that an empty receptacle 39 is replaced by a new receptacle 39 filled with disinfecting solution.

The cover 40, which serves for enclosing the receptacle 39, is removably attached to the forward carrier 29, e.g. by providing a bayonet mount.

The control unit 24 may include a communication module configured to enable a wireless communication with a mobile phone, e.g. via Bluetooth. An app may be provided which runs on the mobile phone and which allows the control of the device. For instance, using the app, the number of revolutions that the body part 20 and with it the spraying head 30 should make can be defined and subsequently the device can be activated.

When activated, the body part 20 begins to rotate with respect to the base 10 and the pump 27 causes disinfecting solution to be sucked from the receptacle 39 and transported to the spraying head 30, from where the disinfecting solution is discharged in small droplets to the outside.

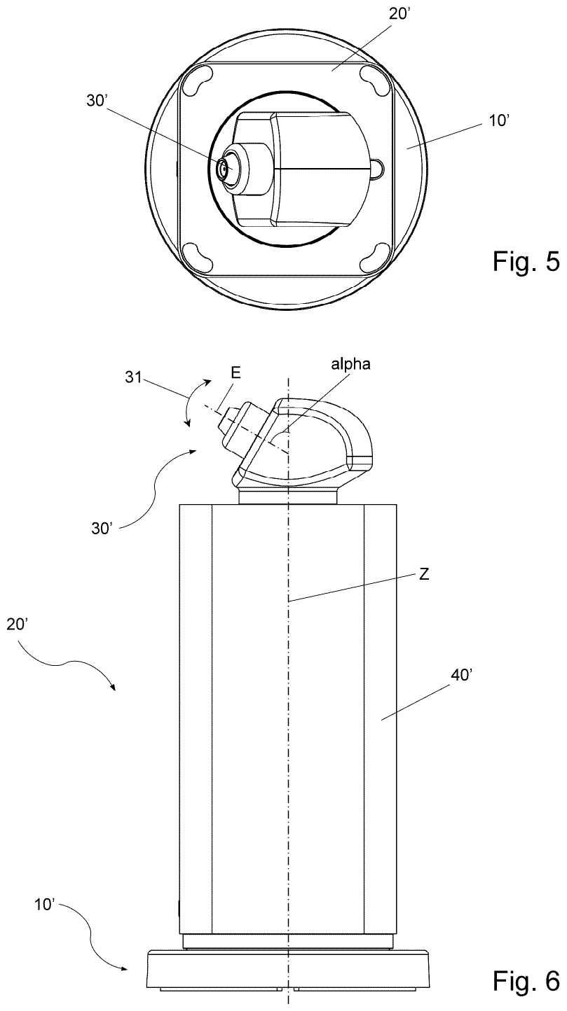

FIGS. 5 to 8 show a second embodiment of a device for discharging a disinfecting solution to disinfect surfaces. The device of this embodiment is configured to be arranged on a carrier surface arranged horizontally, e.g. on a table, and includes a base 10′ to be arranged on the carrier surface. The device includes a body part 20′ which is configured to be rotatable relative to the base 10′, which is stationary when arranged on the carrier surface. The body part 20′ includes a spraying head 30′ for discharging the disinfecting solution. The spraying head 30′ may be configured such that, in addition to the rotation along the longitudinal axis Z of the body part 20′, it can be swivelled e.g. manually as indicated by the double arrow 31 as indicated in FIG. 6. Thereby, the angle alpha between the longitudinal axis Z and the main discharging direction E of the disinfecting solution can be adjusted if required.

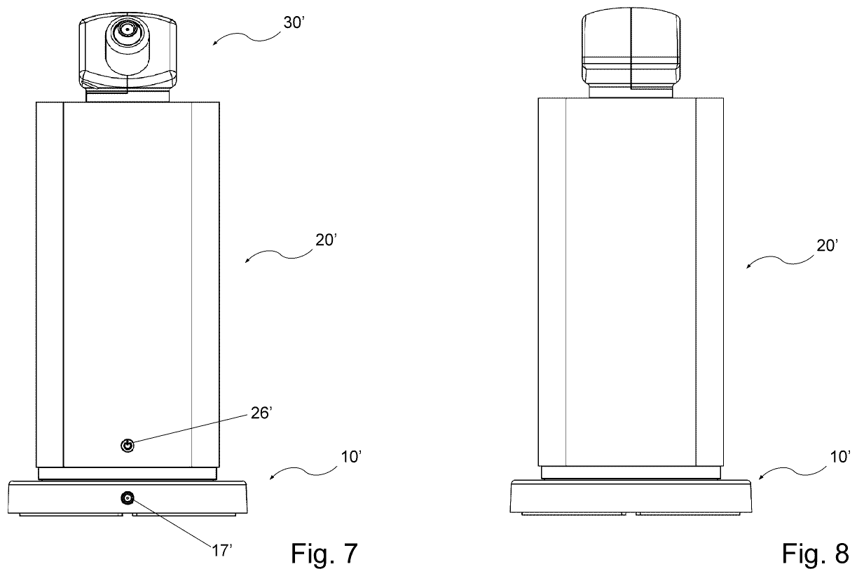

As is apparent from FIG. 7, the device includes a control button 26′ which is arranged in the present embodiment on the body part 20′. The control button 26′ serves for switching on and off the device. Further functions may be activated when operating the control button 26′, e.g. a resetting of the device.

As is further depicted in FIG. 7, the base 10′ includes an electrical connector 17′, e.g. a DC terminal, which can be connected to a power supply unit, e.g. a power supply unit 11 as shown in FIG. 1, to supply the device with energy.

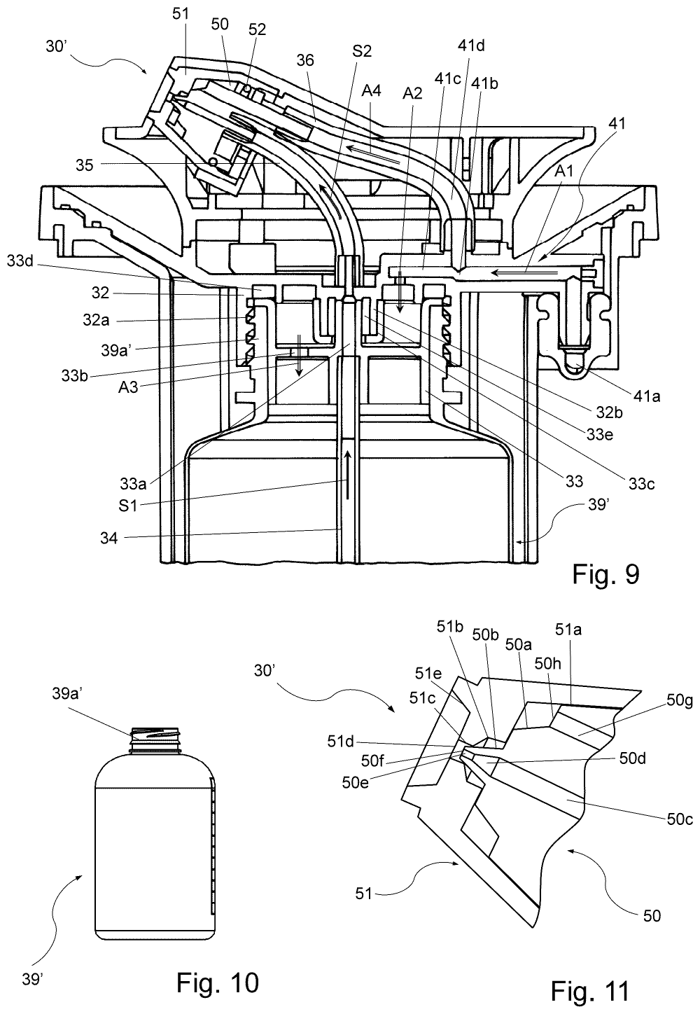

The body part 20′ includes a cover 40′, which is removable to reveal a receptable 39′ containing a disinfecting solution, see FIG. 9.

To allow a rotation of the body part 20′, the base 10′ of the device includes-similar as the components 16, 18 of the first embodiment-a circumferential internal toothing and a bearing, e.g. a ball bearing, arranged coaxially to said internal toothing.

Similar as the components 22, 23, 24, 27 of the first embodiment the body part 20′ includes
a drive, e.g. a stepper motor, with a rotary shaft provided with a drive gear meshing with the internal toothing of the base 10′,
a control unit for controlling the device,
a pump, e.g. a pump for producing high-pressure air.

The control unit may include a communication module configured to enable a wireless communication with a mobile phone, e.g. via Bluetooth.

To reduce unwanted effects from vibrations and/or noise, the pump may be arranged on the body part 20′ by means of dampening elements similar to the components 28a of the first embodiment.

FIG. 10 shows the receptacle 39′ in form of a bottle for receiving the disinfecting solution. The bottle 39′ includes bottleneck 39a′ with a thread which can be screwed into an inner thread 32a formed in a connection port 32 of the body part 20′, see FIG. 9. In the second embodiment, the drive, the control unit and the pump mentioned above are arranged between the base 10′ and the lower part of the receptacle 39′.

When activated, the body part 20′ begins to rotate with respect to the base 1′0 and the pump causes disinfecting solution to be sucked from the receptacle 39′ and transported to the spraying head 30′, from where the disinfecting solution is discharged in small droplets to the outside.

The receptacle 39′ may be configured to be disposable, so that an empty receptacle 39′ can be replaced by a new receptacle 39′ filled with disinfecting solution.

In connection with FIGS. 9 and 11 discharging of the disinfecting solution is explained in more detail.

A plug 33 is inserted into the bottleneck 39a′. The plug 33 includes a through opening 33a, which may be arranged in the centre and to which a suction tube 34 is attached reaching to the bottom of receptacle 39′. The lower end of the suction tube 34 may be provided with a filter (not shown) configured to filter out particles and other impurities in order to prevent a clogging in particular of the spraying head 30′.

The plug 33 includes at least one hole 33b which is arranged laterally to the through opening 33a and which fluidically connects the interior of the receptacle 39′ above the disinfecting solution with a feed channel 41 for pressurized air.

The plug 33 has a central recess 33c which is annular as the outlet end of the wall defining the through opening 33a extends into it.

The connection port 32 includes an annular protrusion 32b in the centre which engages into the central recess 33c of the plug 33.

A gasket 33d is arranged between the top of the plug 33 and the inner part of the connection port 32. The protrusion 32b is provided with a sealing sleeve 33e. The sealing material 33d and 33e allows an airtight connection of the receptacle 39′ when connected to the connection port 32. Thereby, the receptacle 39′ can be pressurized without leakage.

There is provided a connection tube 35 connecting the through opening 33a in the plug 33 with the spraying head 30′.

The feed channel 41 includes a connection end 41a which can be connected to the pump, such as a pump 27 shown in FIG. 4, and a bifurcation 41b to form a first branch 41c leading to the plug 33 and a second branch 41d leading to a connection tube 36.

The spraying head 30′ includes a nozzle with an inner nozzle part 50 and an outer nozzle part 51. The nozzle parts 50 and 51 may be removably connected e.g. by means of a screw connection. To this end, the inner nozzle part 50 may include on its outer surface a thread, which engages into a thread formed on the inner surface of the outer nozzle part 51. To ensure an airtight connection, a sealing ring 52 is arranged between the two nozzle parts 50 and 51.

FIG. 11 shows the construction of the outlet part of the spraying head 30′ in more detail. The outer surface of the inner nozzle part 50 has a tapered form and includes a conical middle portion 50a followed by a conical tip portion 50b which is smaller than the middle portion 50a.

A first channel including a first channel portion 50c, which via a second channel portion 50d merges into a third channel portion 50e, runs through the centre of the interior of the inner nozzle part 50. The first channel portion 50c and third channel portion 50e may have a constant diameter. The diameter of the third channel portion 50e is smaller than the diameter of the first channel portion 50c, such that the second channel portion 50d has a tapering (e.g. conical) shape. The outward end of the third channel portion 50e defines a liquid outlet 50f. The diameter of the liquid outlet 50f is less than 1.00 mm, preferably less than 0.50 mm. Typically, the diameter is in the range 0.30 to 0.40 mm. The diameter may be e.g. 0.36 mm.

A second channel 50g runs laterally to the first channel 50c-50e in the interior of the inner nozzle part 50 and leads to an air outlet 50h.

The inner surface of the outer nozzle part 51 has a tapering shape and includes a first surface portion 51a, which via a second surface portion 51b merges into a third surface portion 51c. The first surface portion 51a may be e.g. conical. The second surface portion 51b tapers towards the third surface portion 51c. The third surface portion 51c defines a passage in which the liquid outlet 50f is arranged and which merges into a spray outlet 51d. A fourth surface portion 51e of the outer nozzle part 51 follows the spray outlet 51d and forms a recess in the outer nozzle part 51. The minimum diameter of this recess is greater than the maximum diameter of the third surface portion 51c and may be greater than the maximum diameter of second surface portion 51b.

The diameter of the spray outlet 51d is less than 4 mm, preferably less than 3 mm. The diameter may be e.g. 2.0 mm.

In use, the pump 27 produces a flow of high-pressure air, which is fed via the connection end 41a into the feed channel 41 and guided towards the bifurcation 41b as indicated by the arrow A1 in FIG. 9. Part of the air flows via the first branch 41c towards the plug 33 (see arrow A2) and passes via hole 33b into the receptacle 39′ (see arrow A3), where the disinfecting solution is pressurized. This causes disinfecting solution to rise in the suction tube 34 as indicated by arrow S1 and to be transported via the through opening 33a and the connection tube 35 towards the spraying head 30′ (see arrow S2).

The other part of the air in the feed channel 41 is diverted at the bifurcation 41b into the second branch 41d and guided via the connection tube 36 towards the spraying head 30′ (see arrow A4).

Referring to FIG. 11, the disinfecting solution in the connection tube 35 is pressed via the channel 50c-50e out of the liquid outlet 50f, where it is mixed with air flowing via the connection tube 36 and the second channel 50g out of the air outlet 50h and subsequently through the space between the inner nozzle part 50 and the outer nozzle part 51 towards the channel portion defined by the third surface portion 51c. In said channel portion, the disinfecting solution is mix with pressurized air. The disinfecting solution is divided into small droplets. The droplets ejected from the spraying head 30′ are small enough so that they are suspended in the air ejected out of the spray outlet 51d. Thus, the spraying head 30′ serves as an atomizer for the disinfecting solution. The initial speed of the ejected droplets of disinfecting solution is large enough so that they are ejected up to the desired distance. This distance measured from the spray outlet 51d may be at least 1 m, preferably at least 2 m.

When the pump 27 is switched off, the compressed air in the receptacle 39′ can escape via the components 33b, 41c, 41d out of the air outlet 50h so that the pressure in the receptacle 39′ is normalized and transporting of disinfecting solution towards the spraying head 30′ is stopped.

The embodiments of the device described so far are configured to be arranged on a horizontal surface. It is conceivable to configure the base 10, 10′ such that it can be mounted on the wall. The device may be configured such that the spray head is rotatable over an angle range of at least 90 degrees and less than 360 degrees, e.g. 180 degrees. In use, the spray head is rotated back and forth.

The device described herein can be used for various applications, in particular in the medical sector, such as in hospitals and in medical and dental practices, as well as hotels, restaurants, public rooms, public transportation, aviation, classrooms, offices, private homes and other locations. The device may be configured such that the maximum range up to which the disinfecting solution can be discharged from the spray head is at least 0.5 m, preferably at least 1 m, most preferably at least 2 m, e.g. 2.5 m.

From the preceding description, many modifications are available to the skilled person without departing from the scope of the invention, which is defined in the claims.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

The device and the spray head may be configured such that another liquid than a disinfecting solution may be discharged in droplets suspended in the air.

1. A device for discharging a disinfecting solution to disinfect surfaces, the device being configured to be arrangeable on a carrier surface and comprising:
a receptacle for receipt of the disinfecting solution, the receptacle being exchangeably arranged;
a housing enclosing the receptacle;
a spray head configured to discharge disinfecting solution from the receptacle; and
a pumping equipment for transporting disinfecting solution from the receptacle to the spray head and for discharging the disinfecting solution from the spray head.

2. The device according to claim 1, further comprising a base part arrangeable on the carrier surface and a rotary part, which includes the spray head and which is configured to be rotatable with respect to the base part.

3. The device according to claim 2, wherein the base part comprises an internal toothing and the rotary part comprises a drive gear meshing with the internal toothing.

4. The device according to claim 2 or 3, wherein the rotary part comprises at least one of the receptacle and an electric drive.

5. The device according to claim 2, further comprising an electric transmission equipment configured to supply electrical power from the base part to the rotary part.

6. The device according to claim 5, wherein the electric transmission comprises a rotary electric slip ring.

7. The device according to claim 1, wherein the receptacle comprises a bottle including at least one of a threaded bottleneck and a bottleneck with a plug, the plug comprising a through opening for extracting disinfecting solution and at least one hole for pressurizing the disinfecting solution in the bottle.

8. The device according to claim 1, further comprising a connection port configured to be connected to the receptacle.

9. The device according to claim 8, wherein the connection port is configured to be connected to the receptacle by a screw connection.

10. The device according to claim 8, wherein the connection port comprises a sealing equipment configured to provide an airtight connection with the receptacle.

11. The device according to claim 8, wherein the connection port is arranged on top of the receptacle.

12. The device according to claim 1, wherein the pumping equipment comprises a pump to produce pressurized air.

13. The device according to claim 1, further comprising a feed channel with a bifurcation to guide pressurized air to the receptacle and to the spray head.

14. The device according to claim 1, wherein the spray head comprises a first channel for conducting disinfecting solution to a liquid outlet and a second channel for conducting pressurized air to the liquid outlet to mix it with the disinfecting solution ejecting out of the liquid outlet.

15. The device according to claim 14, wherein the liquid outlet is part of a first nozzle configured to receive the disinfecting solution from the receptacle and arranged in a second nozzle, in which pressurized air can be added.

16. The device according to claim 1, wherein the spray head comprises an inner nozzle part for receiving and ejecting disinfecting solution and an outer nozzle part for receiving pressurized air and mixing it to the ejected disinfecting solution, wherein the inner nozzle part and the outer nozzle part are detachably connected to each other.

17. The device according to claim 16, wherein a seal is arranged between the inner nozzle part and the outer nozzle.

18. The device according to claim 16, wherein the inner nozzle part and the outer nozzle are detachably connected by means of a screw connection.

19. The device according to claim 1, further comprising a communication module configured to enable a wireless communication with a mobile phone for controlling the device.

20. The device according to claim 1, wherein the housing comprises a removable cover enabling an access to the receptacle for a replacement.

21. The device according to claim 1, further comprising a power supply unit configured to supply the device with power and connectable to the mains via a cable.

22. A spray head for a device for discharging a liquid in atomized form, the spray head comprising:
an inner nozzle part including a first channel for conducting liquid to a liquid outlet and a second channel for conducting pressurized air to an air outlet; and
an outer nozzle part including a spray outlet and enclosing the inner nozzle part to form a space between the air outlet und the spray outlet, the liquid outlet being arranged in the space to mix liquid ejecting out of the liquid outlet with pressurized air.

23. The spray head according to claim 22, wherein the inner nozzle part comprises an outer surface portion, which converges to a tip portion including the liquid outlet.

24. The spray head according to claim 22, wherein the outer nozzle part comprises an inner surface portion, which converges to a channel portion including the spray outlet.

25. The spray head according to any one of claim 22, wherein the first channel is arranged centrally in the inner nozzle part and the second channel is arranged radially offset to the first channel.

26. The spray head according to any one of claim 22, wherein the inner nozzle part and the outer nozzle are detachably connected.

27. The spray head according to any one of claim 22, wherein a seal is arranged between the inner nozzle part and the outer nozzle.

28. The spray head according to any one of claim 22, wherein the liquid outlet of the inner nozzle part has a first diameter and the spray outlet of the second nozzle part has a second diameter and wherein at least one of the following sizes applies:
the first diameter is less than 1.0 mm;
the first diameter is less than 0.5 mm;
the first diameter is in the range of 0.3 to 0.4 mm;
the second diameter is less than 4 mm;
the second diameter is less than 3 mm;
the distance between the liquid outlet and the spray outlet is less than 4 mm;
the distance between the liquid outlet and the spray outlet is less than 3 mm.

29. The spray head according to any one of claim 22, wherein a recess is arranged on the outside of the outer nozzle part, the spray outlet being arranged in the recess.

30. A device for discharging a liquid, the device being configured to be arrangeable on a carrier surface and comprising:
a receptacle for receipt of the liquid, the receptacle being exchangeably arranged;
a housing enclosing the receptacle;
a spray head configured to discharge liquid from the receptacle; and
a pumping equipment for transporting liquid from the receptacle to the spray head and for discharging the disinfecting solution from the spray head.

31. A device for discharging a liquid, the device comprising a spray head according to claim 22.
